(19) United States
(12) Patent Application Publication
(10) Pub. No.: US 2002/0172720 A1
(43) Pub. Date: Nov. 21, 2002

(54) Control of pseudomonas aeruginosa

(76) Inventor: Christopher J. Nalepa, Baton Rouge, LA (US)

Correspondence Address: Mr. Philip M. Pippenger, Patent & Trademark Division, Albemarle Corporation, 451 Florida Street, Baton Rouge, LA, 70801-1765, US

(21) Appl. No.: 09/819,153

(22) Filed: Mar. 27, 2001

(51) Int. Cl. A61K 33/32; A61K 33/14

(52) U.S. Cl. 424/641; 424/661; 424/673; 514/389

(57) ABSTRACT

Pseudomonas aeruginosa biofilm on a surface is eradicated or at least reduced using a microbiocidally effective amount of at least one of the following bromine-based microbiocides:

i) an alkali or alkaline earth metal bromide salt or zinc bromide activated by a water-soluble alkali or alkaline earth metal hypochlorite,

ii) a water-soluble N,N′-bromochloro-5,5-dialkylhydantoin, and/or

iii) a water-soluble active bromine product formed by mixing (a) bromine, bromine chloride, a mixture of bromine chloride and bromine or a combination of bromine and chlorine in which the molar amount of chlorine does not exceed the molar amount of bromine, and (b) a water-soluble source of sulfamate anion, in (c) an aqueous medium, which aqueous medium is adjusted to a pH of at least 12 either during or after such mixing.

Pseudomonas aeruginosa is a strain of bacteria which can form biofilm on surfaces in contact with water. As is well recognized in the art, biofilms are more resistant to the action of microbiocides than are planktonic bacteria i.e., bacteria which are not attached to a surface. In this connection, Pseudomonas aeruginosa is an especially tenacious biofilm former in that it is highly resistant to chemical removal. In addition, Psuedomonas aeruginosa has the ability of protecting itself against the action of microbiocides by forming extracellular polysaccharide slime layers which resist penetration of water-borne microbiocides. The rate at which such slime layers is formed is quite rapid in the case of Pseudomonas aeruginosa—highly resistant slime layers can be formed on a clean surface in less than one week. In addition, biofilms of Psuedomonas aeruginosa typically harbor dangerous pathogens, and cause damage to the surfaces to which they have become attached.

An object of this invention is to provide an effective way of controlling Pseudomonas aeruginosa even after it has formed and extracellular polysaccharide slime layer.

Pursuant to this invention, it has been found that certain bromine containing biocides are effective in controlling Pseudomonas aeruginosa on water exposed surfaces even after an extracellular polysaccharide defensive layer has been produced by the bacteria species.

The bromine based microbiocides used pursuant to this invention are (i) at least one alkali or alkaline earth metal bromide salt or zinc bromide activated by a water-soluble alkali or alkaline earth metal hypochlorite, (ii) at least one water-soluble N,N′-bromochloro-5,5-dialkylhydantoin, and/or (iii) at least one water-soluble active bromine product formed by mixing (a) bromine, bromine chloride, a mixture of bromine chloride and bromine or a combination of bromine and chlorine in which the molar amount of chlorine does not exceed the molar amount of bromine, and (b) a water-soluble source of sulfamate anion, in (c) an aqueous medium, which aqueous medium is adjusted to a pH of at least 12 and preferably at least 13 either during or after such mixing. Microbiocides of this type (i) can also contain a halogen stabilizer such as a source of sulfamate anion. One such product is available commercially under the trade designation Stabrex® biocide (Nalco Corporation).

In forming the microbiocides of type (i) use can be made of one or a mixture of such Group 1 or 2 metal bromide salts as for example sodium bromide, potassium bromide, lithium bromide, magnesium bromide, calcium bromide, or like substances. As noted above, zinc bromide can also be used either alone or in combination with any of the foregoing. Aqueous solutions of sodium bromide adapted for use in forming such microbiocides are available in the marketplace under the trade designation Sanibrom® 40 biocide or Sanibrom® 43 (Albemarle Corporation). Suitable alkali or alkaline earth metal hypochlorites which can be used include sodium hypochlorite, potassium hypochlorite, lithium hypochlorite, magnesium hypochlorite, calcium hypochlorite or the like. These components are mixed together in proportions such that there is at least one equivalent of hypochlorite per equivalent of bromide. An excess of up to about 10 equivalents of hypochlorite per equivalent of bromide is feasible.

Methods for the production of microbiocides of type (ii) above generally involve brominating and chlorinating a suitable 5,5,-dialklyhydantoin using proportions of these halogens sufficient to produce a product containing essentially one atom of bromine and one atom of chlorine per molecule. Suitable bromochlorohydantoins include such compounds as N,N′-bromochloro-5,5-dimethylhydantoin, N,N′-bromochloro-5-ethyl-5-methylhydantoin, N,N′-bromochloro-5-propyl-5-methylhydantoin, N,N′-bromochloro-5-isopropyl-5-methylhydantoin, NN′-bromochloro-5-butyl-5-methylhydantoin, NN′-bromochloro-5-isobutyl-5-methylhydantoin, N,N′-bromochloro-5-sec-butyl-5-methylhydantoin, N,N′-bromochloro-5-tert-butyl-5-methylhydantoin, and mixtures of any two or more of the foregoing. N,N′-bromochloro-5,5-dimethylhydantoin is available commercially under the trade designation Bromicide® biocide (Great Lakes Chemical Corporation). Another suitable bromochlorohydantoin mixture is composed predominantly of N,N′-bromochloro-5,5-dimethylhydantoin together with a minor proportion by weight of 1,3-dichloro-5-ethyl-5-methylhydantoin. A mixture of this latter type is available in the marketplace under the trade designation Dantobrom® biocide (Lonza Corporation).

Microbiocides of type (iii) above can be prepared as described, for example, in U.S. Pat. No. 6,068,861, all disclosure of which is incorporated herein by reference. A concentrated solution of sulfamate-stabilized bromine chloride is available in the marketplace under the trade designation Stabrom® 909 biocide (Albemarle Corporation).

In the practice of this invention the above bromine-based microbiocide can be applied to the biofilm either by adding the microbiocide to the aqueous medium in contact with the biofilm or, in the case of surfaces which can be removed from the aqueous medium with which they are normally in contact, by contacting the biofilm with an aqueous solution of the microbiocide. In either case, the amount of microbiocide used is a microbiocidally effective amount which to some extent will vary depending upon the identity of the microbiocide being used and the age of the biofilm colony. In this connection, the greater the age of a colony of Pseudomonas aeruginosa biofilm the more resistant it is to the action of the microbiocide and thus in general the greater should be the dosage level of the microbiocide. Generally speaking, biocidally effective concentrations in water whether dissolved in the water by any suitable means or be direct application from an aqueous solution should be at least in the range of about 2 to about 200 ppm (wt/wt) and preferably in the range of about 5 to about 20 ppm (wt/wt). Departures from the foregoing ranges can be made whenever deemed necessary or desirable under the particular circumstances at hand, and such departures are within the scope of this invention. The duration of the exposure to the aqueous microbiocidal medium used pursuant to this invention can be as little as a few minutes up to hours or even days, depending upon the age of the colony of Pseudomonas aeruginosa biofilm. That is, the older the colony, the longer should be the contact time.

The efficacy of this invention in eradicating (i.e. controlling) Pseudomonas aeruginosa biofilm of sufficient age such that it had developed an extracellular polysaccharide protective slime layer, was shown by a series of standard tests performed on our behalf at MBEC Biofilm Technologies, Inc., Calgary, Canada. The test procedure, developed at the University of Calgary, utilizes a device which allows the growth of 96 identical biofilms under carefully controlled conditions. The device consists of a two-part vessel comprised of an upper plate containing 96 pegs that seals against a bottom plate. The bottom plate can consist of either a trough (for biofilm growth) or a standard 96-well plate (for biocide challenge). The biofilms develop on the 96 pegs. The device has been used as a general method for evaluating the efficacy of antibiotics and biocides towards biofilms. See in this connection H. Ceri, et al., “The MBEC Test: A New In Vitro Assay Allowing Rapid Screening for Antibiotic Sensitivity of Biofilm”, Proceedings of the ASM, 1998,89,525; Ceri, et al., “Antifungal and Biocide Susceptibility testing of Candida Biofilms using the MBEC Device”, Proceedings of the Interscience Conference on Antimicrobial Agents and Chemotherapy, 1998, 38, 495; and H. Ceri, et al., “The Calgary Biofilm Device: A New Technology for the Rapid Determination of Antibiotic Susceptibility of Bacterial Biofilms”, Journal of Clinical Microbiology, 1999, 37, 1771-1776.

Six biocide systems were evaluated using the above test procedure and test equipment. Five of these systems were oxidizing biocides, viz., chlorine (from NaOCl), bromine (from NaOCl+NaBr), bromine and chlorine (from BCDMH), bromine (from sulfamate stabilized bromine chloride), and chlorine (from trichloroisocyanuric acid), all expressed as bromine (Br₂) in mg/L, so that all test results were placed on the same basis. The sixth biocide was glutaraldehyde, a non-oxidizing biocide.

These biocide systems were used to challenge biofilms of Pseudomonas aeruginosa (ATCC 15442). This is a Gram (−) bacterium which is known to rapidly develop an extracellular polysaccharide slime layer, and is found in industrial and recreational water systems. See in this connection J. W. Costerton and H. Anwar, “Pseudomonas aeruginosa: The Microbe and Pathogen”, in Pseudomonas aeruginosa Infections and Treatment, A. L. Baltch and R. P. Smith editors, Marcel Dekker publishers, New York, 1994.

In the Table the MBEC (minimum biofilm eradication concentration) results presented are for the one-hour biocide contact time used in the test. The Psuedomonas aeurginosa colony was allowed to grow in contact with a nutrient solution for seven days. The values given for the halogen containing biocides are expressed in terms of mg/L of bromine as Br₂. The data on the glutaraldehyde is in terms of mg/L as active ingredient. The data indicate that the bromine-based microbiocides used pursuant to this invention were surpisingly more effective against the Pseudomonas aeruginosa biobilm than the chlorine-based microbiocides, as well as the glutaraldehyde biocide.

TABLE
EFFECTIVENESS AGAINST PSEUDOMONAS AERUGINOSA
BIOFILM

| | MBEC, ppm as | |
|---|---|---|
| | Br₂, on 7-day | |
| Biocode System | old biofilm | MBEC, avg. |
| Bromine from stabilized BrCl | 5, 5 | 5 |
| Bromine (from NaOCL + NaBr) | 5, 10 | 7.5 |
| Bromine and chlorine (from BCDMH) | 5, 10 | 7.5 |
| Chlorine (from NaOCl) | 20, 20 | 20 |
| Chlorine (from Trichloroisocyanuric acid) | 20, 20 | 20 |
| Glutaraldehyde | 100, >200 | 200 (est.) |

Compounds referred to by chemical name or formula anywhere in this document, whether referred to in the singular or plural, are identified as they exist prior to coming into contact with another substance referred to by chemical name or chemical type (e.g., another component, a solvent, or etc.). It matters not what preliminary chemical changes, if any, take place in the resulting mixture or solution, as such changes are the natural result of bringing the specified substances together under the conditions called for pursuant to this disclosure. Also, even though the claims may refer to substances in the present tense (e.g., “comprises”, “is”, etc.), the reference is to the substance as it exists at the time just before it is first contacted, blended or mixed with one or more other substances in accordance with the present disclosure.

All documents referred to herein are incorporated herein in toto as if fully set forth in this document.

This invention is susceptible to considerable variation within the spirit and scope of the appended claims.

That which is claimed is:

1. A method of eradicating or at least reducing Pseudomonas aeruginosa biofilm on a surface, said biofilm containing extracellular polysaccharide, which method comprises contacting said biofilm with an aqueous medium or water into which has been introduced a microbiocidally effective amount of at least one of the following bromine-based microbiocides:
i) at least one alkali or alkaline earth metal bromide salt or zinc bromide activated by a water-soluble alkali or alkaline earth metal hypochlorite,
ii) at least one water-soluble N,N′-bromochloro-5,5-dialkylhydantoin, and/or
iii) at least one water-soluble active bromine product formed by mixing (a) bromine, bromine chloride, a mixture of bromine chloride and bromine or a combination of bromine and chlorine in which the molar amount of chlorine does not exceed the molar amount of bromine, and (b) a water-soluble source of sulfamate anion, in (c) an aqueous medium, which aqueous medium is adjusted to a pH of at least 12 either during or after such mixing, said contacting being for one or more periods long enough to eradicate or at least reduce said biofilm.

2. A method of claim 1 wherein said biofilm comprises at least one additional pathogenic organism.

3. A method of claim 2 wherein said bromine-based microbiocide used is formed by mixing sodium bromide and sodium hypochlorite in water.

4. A method of claim 3 wherein the sodium bromide and sodium hypochlorite are mixed in water to which a source of sulfamate anion is included.

5. A method of claim 2 wherein said bromine-based microbiocide used is at least one water-soluble N,N′-bromochloro-5,5-dialkylhydantoin.

6. A method of claim 5 wherein said N,N′-bromochloro-5,5-dialkylhydantoin is N,N′-bromochloro-5,5-dimethylhydantoin.

7. A method of claim 5 wherein said N,N′-bromochloro-5,5-dialkylhydantoin is a mixture of N,N′-bromochloro-5,5-dimethylhydantoin and 1,3-dichloro-5-ethyl-5-methylhydantoin.

8. A method of claim 2 wherein said bromine-based microbiocide used is at least one water-soluble active bromine product formed by mixing (a) bromine, bromine chloride, a mixture of bromine chloride and bromine or a combination of bromine and chlorine in which the molar amount of chlorine does not exceed the molar amount of bromine, and (b) a water-soluble source of sulfamate anion, in (c) an aqueous medium, which aqueous medium is adjusted to a pH of at least 12 either during or after such mixing.

9. A method of claim 8 wherein said product is a sulfamate stabilized bromine chloride.

10. A method of eradicating or at least reducing Pseudomonas aeruginosa biofilm on a surface, said biofilm containing extracellular polysaccharide, which method comprises adding to the aqueous medium in contact with said biofilm a microbiocidally effective amount of at least one of the following bromine-based microbiocides:
i) at least one alkali or alkaline earth metal bromide salt or zinc bromide activated by a water-soluble alkali or alkaline earth metal hypochlorite,
ii) at least one water-soluble N,N′-bromochloro-5,5-dialkylhydantoin, and/or
iii) at least one water-soluble active bromine product formed by mixing (a) bromine, bromine chloride, a mixture of bromine chloride and bromine or a combination of bromine and chlorine in which the molar amount of chlorine does not exceed the molar amount of bromine, and (b) a water-soluble source of sulfamate anion, in (c) an aqueous medium, which aqueous medium is adjusted to a pH of at least 12 either during or after such mixing, said contacting being for one or more periods long enough to eradicate or at least reduce said biofilm.

11. A method of claim 10 wherein said biofilm comprises at least one additional pathogenic organism.

12. A method of claim 11 wherein said bromine-based microbiocide used is formed by mixing sodium bromide and sodium hypochlorite in water.

13. A method of claim 12 wherein the sodium bromide and sodium hypochlorite are mixed in water to which a source of sulfamate anion is included.

14. A method of claim 11 wherein said bromine-based microbiocide used is at least one water-soluble N,N′-bromochloro-5,5-dialkylhydantoin.

15. A method of claim 14 wherein said N,N′-bromochloro-5,5-dialkylhydantoin is N,N′-bromochloro-5,5-dimethylhydantoin.

16. A method of claim 14 wherein said N,N′-bromochloro-5,5-dialkylhydantoin is a mixture of N,N′-bromochloro-5,5-dimethylhydantoin and 1,3-dichloro-5-ethyl-5-methylhydantoin.

17. A method of claim 11 wherein said bromine-based microbiocide used is at least one water-soluble active bromine product formed by mixing (a) bromine, bromine chloride, a mixture of bromine chloride and bromine or a combination of bromine and chlorine in which the molar amount of chlorine does not exceed the molar amount of bromine, and (b) a water-soluble source of sulfamate anion, in (c) an aqueous medium, which aqueous medium is adjusted to a pH of at least 12 either during or after such mixing.

18. A method of claim 17 wherein said product is a sulfamate stabilized bromine chloride.
